(19) United States
(12) Patent Application Publication
(10) Pub. No.: US 2024/0238261 A1
(43) Pub. Date: Jul. 18, 2024

(54) METHOD OF TREATING ESSENTIAL TREMOR

(71) Applicant: Cavion, Inc., Palo Alto, CA (US)

(72) Inventors: Margaret S. Lee, Middleton, MA (US); Michelle Gilbert Baladi, Palo Alto, CA (US)

(21) Appl. No.: 18/563,101

(22) PCT Filed: May 23, 2022
(86) PCT No.: PCT/US22/72510

§ 371 Date: Nov. 21, 2023

Related U.S. Application Data
(60) Provisional application No. 63/192,535, filed on May 24, 2021.

(51) Int. Cl. A61K 31/4412; A61K 9/00; A61P 25/16

(52) U.S. Cl. CPC A61K 31/4412; A61K 9/0053; A61P 25/16

(57) ABSTRACT

Provided herein are methods of treating a movement disorder in an individual in need thereof, comprising administering to the individual an oral dosage form of CX-8998 or a pharmaceutically acceptable salt thereof.

### CROSS-REFERENCE TO RELATED APPLICATIONS

This application claims the benefit of and the priority to U.S. Provisional Patent Application No. 63/192,535 filed May 24, 2021, the disclosure of which is hereby incorporated by reference in its entirety.

### BACKGROUND

Essential Tremor (ET) is among the most prevalent of all movement disorders in adults. In a 2010 meta-analysis, Louis et al. (1998 Movement Disorders. 13(1):5-10) estimated the pooled prevalence (all ages) to be 0.9%, with statistically significant heterogeneity across studies (12=99%, p<0.001). The prevalence in adults ≥65 years old was estimated to be 4.6% (Louis and Ferreira, 2010 Mov Disord. 25(5): 534-541). While ET does not shorten life expectancy, its impact on the patient's ability to perform activities of daily living (ADLs, such as writing and eating) at home and in the work place negatively affects quality of life, social interactions, and mental status (Lorenz et al., 2006 Mov Disord. 21(8): 1114-1118; Louis et al., 2015 Parkinsonism Relat Disord. 21(7): 729-735: George et al., 1994 Psychosomatics. 35(6):520-523; and Zesiewicz et al., 2011 Neurology. 77(19): 1752-1755). It is increasingly recognized that ET is not a monosymptomatic disorder (Bermejo-Pareja, 2011 Nature Reviews Neurology. 7(5):273-282). Effects on cognitive functions are heterogeneous and include impairments in attention, executive function, verbal fluency, visuospatial functioning, memory, and working memory (Bermejo-Pareja et al., 2012 “V. Cognitive Features of Essential Tremor: A Review of the Clinical Aspects and Possible Mechanistic Underpinnings,” pages 2:02-74-541-1 in Tremor and Other Hyperkinetic Movements (Louis, ed.) 2012). Sleep disturbances and fatigue are also more common in patients with ET than in their age-matched controls (Chandran et al., 2012 Acta Neurol Scand. 125:332-7). Essential tremor typically worsens over time and can be severe in some people. It is a significant disability that affects many activities of daily living and can be a source of social embarrassment, phobia, depression & anxiety.

CX-8998 is a first-in-class T-type calcium channel antagonist and is the most advanced late-stage small molecule currently in development for essential tremor. CX-8998 was evaluated in T-CALM (Tremor-Caᵥ₃ modulation), a proof of concept, Phase 2 double-blind, placebo-controlled clinical study of 95 patients with essential tremor conducted at 25 sites across the United States. Patients were randomized to one of two treatment arms, receiving either placebo or CX-8998. Significant improvements across a number of clinical measures, including the investigator-rated The Essential Tremor Rating Assessment Scale (TETRAS) Performance Subscale (P=. 017), TETRAS-Activities of Daily Living (TETRAS-ADL: P=. 049), TETRAS total score (P=. 007) and Clinician Global Impression of Improvement (P=. 001), were observed in patients with essential tremor who were treated with CX-8998 (Papapetropoulos et al. presented at: 2019 American Academy of Neurology Annual Meeting, Philadelphia, PA).

### SUMMARY

Provided here are methods of treating a movement disorder. Various embodiments are contemplated herein. For example in Embodiment 1, provided is a method of treating a movement disorder in an individual in need thereof, comprising: a) administering to the individual a first dose of an oral dosage form of CX-8998 or a pharmaceutically acceptable salt thereof, wherein the first dose comprises about 5 mg of CX-8998 and wherein the first dose is administered orally to the individual once daily (QD) on each day of week 1: b) administering to the individual a second dose of an oral dosage form of CX-8998 or a pharmaceutically acceptable salt thereof, wherein the second dose comprises about 10 mg of CX-8998 and wherein the second dose is administered orally to the individual once daily (QD) on each day of week 2; and c) optionally administering to the individual a third dose of an oral dosage form of CX-8998 or a pharmaceutically acceptable salt thereof, wherein the third dose comprises about 20 mg of CX-8998 and wherein the third dose is administered orally to the individual once daily (QD) on each day of week 3.

Embodiment 2: The method of Embodiment 1, wherein the method comprises a) administering the first dose to the individual once daily (QD) on each day of week 1: b) administering the second dose to the individual once daily (QD) on each day of week 2; and c) maintaining the administration of the 10 mg of CX-8998 to the individual once daily (QD) for a period of time following the last dose administered in week 2.

Embodiment 3: The method of Embodiment 1, wherein the method comprises a) administering the first dose to the individual once daily (QD) on each day of week 1: b) administering the second dose to the individual once daily (QD) on each day of week 2: c) administering the third dose to the individual once daily (QD) on each day of week 3; and d) maintaining the administration of the 20 mg of CX-8998 to the individual once daily (QD) for a period of time following the last dose administered in week 3.

Embodiment 4: The method of Embodiment 1, wherein the method comprises a) administering the first dose to the individual once daily (QD) on each day of week 1: b) administering the second dose to the individual once daily (QD) on each day of week 2: c) administering the third dose to the individual once daily (QD) on each day of week 3; and d) administering to the individual a fourth dose of an oral dosage form of CX-8998 or a pharmaceutically acceptable salt thereof, wherein the fourth dose comprises about 30 mg of CX-8998 and wherein the fourth dose is administered orally to the individual once daily (QD) on each day of week 4.

Embodiment 5: The method of Embodiment 4, wherein the method comprises maintaining the administration of the 30 mg of CX-8998 to the individual once daily (QD) for a period of time following the last dose administered in week 4.

Embodiment 6: The method of any one of Embodiments 2, 3, or 5, wherein the period of time is a period of at least 1-4 weeks, 6 months, or 1 year or more.

Embodiment 7: The method of Embodiment 3, wherein the individual experiences no severe adverse event (AE) by the last day of week 2 after the first dose is administered, wherein the adverse event is selected from the group consisting of headache, lightheadedness, dizziness, somnolence, lethargy, impaired concentration, vivid dreams, euphoric mood, elevated mood, racing thoughts, visual hallucination, and syncope.

Embodiment 8: The method of Embodiments 3 or 7, wherein the individual experiences mild adverse event (AE), moderate AE, or no AE by the last day of week 2 after the first dose is administered.

Embodiment 9: The method of Embodiment 4, wherein the individual experiences no severe adverse event (AE) by the last day of week 3 after the first dose is administered, wherein the adverse event is selected from the group consisting of headache, lightheadedness, dizziness, somnolence, lethargy, impaired concentration, vivid dreams, euphoric mood, elevated mood, racing thoughts, visual hallucination, and syncope.

Embodiment 10: The method of Embodiments 4 or 9, wherein the individual experiences mild adverse event (AE), moderate AE, or no AE by the last day of week 3 after the first dose is administered.

Embodiment 11: The method of any one of Embodiments 1-10, wherein the movement disorder is essential tremor, epilepsy, or Parkinson's disease.

Embodiment 12: The method of any one of Embodiments 1-11, wherein the individual is diagnosed with moderate to severe essential tremor.

Embodiment 13: The method of any one of Embodiments 1-12, wherein the individual is diagnosed with severe essential tremor.

Embodiment 14: The method of any one of Embodiments 1-13, wherein the individual is an adult.

Embodiment 15: The method of any one of Embodiments 1-14, wherein the oral dosage form is administered to the individual in a fasted state.

Embodiment 16: The method of any one of Embodiments 1-15, wherein the oral dosage form is administered to the individual in the morning or within about 4 hours after waking.

### DETAILED DESCRIPTION

### Definitions

As used herein, “treatment” or “treating” is an approach for obtaining beneficial or desired clinical results.

As used herein, “pharmaceutically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

As used herein, “an oral dosage form” refers to any formulation suitable for oral administration.

As used herein, “individual” refers to a mammal (e.g., a human) in need of treatment. “Individual”, “participant” and “patient” are used interchangeably herein. The term “adult” refers to an individual 18 years of age or older. In certain embodiments, the individual is a senior adult. e.g., 65 years or older. The term “juvenile” refers to an individual 12 to less than 18 years of age.

As used herein, “therapeutically effective amount” refers to an amount of a compound or a combination therapy sufficient to produce a desired therapeutic outcome. As is understood in the art, a therapeutically effective amount may be in one or more doses, i.e., a single dose or multiple doses may be required to achieve the desired treatment endpoint.

Reference to “about” a value or parameter herein includes (and describes) variations that are directed to that value or parameter per se.

The singular forms “a,” “or,” and “the” include plural referents unless the context clearly dictates otherwise.

The term “comprise” or variations such as “comprises” or “comprising”, will be understood to imply the inclusion of a stated element, integer or step, or group of elements, integers or steps, but not the exclusion of any other element, integer or step, or group of elements, integers or steps. Embodiments described herein include “consisting” and/or “consisting essentially of” aspects.

### CX-8998

CX-8998, also known as MK-8998, is a highly selective voltage-activated calcium channel (Caᵥ) antagonist that shows low nanomolar potency against all three Caᵥ₃ isoforms and >100-fold selectivity against other ion channel targets. The free base of CX-8998 is described chemically as (R)-2-(4-Isopropylphenyl)-N-(1-(5-(2,2,2-Trifluoroethoxy)pyridin-2-yl)ethyl)acetamide having the structural formula:

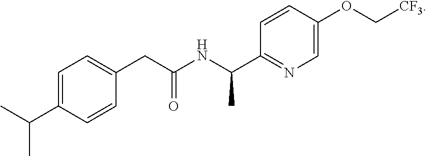 [SMILES: CC(C)c1ccc(CC(=O)N[C@H](C)c2ccc(OCC(F)(F)F)cn2)cc1]

CX-8998 is disclosed in U.S. Pat. No. 7,875,636B2 titled “Pyridyl Amide T-type Calcium Channel Antagonists”, the entirety of which is incorporated herein by reference. Description of CX-8998 and method of making CX-8998 can be found in, e.g., Example 16 in column 39, of the above referenced patent.

CX-8998 has been shown to dose-dependently reduce tremor, absence seizures, and pain in nonclinical models: see, e.g., Papapetropoulos et al. Neurology 2018: 90 (15 Suppl), the entirety of which is incorporated herein by reference. CX-8998 has been undergoing clinical evaluation for the treatment of essential tremor and for the treatment of generalized epileptic syndrome with absence seizure (see, e.g., NCT 03101241 and NCT03406702).

CX-8998 may be in the form of a salt, such as CX-8998 hydrochloride salt. CX-8998 hydrochloride salt is an off-white crystalline powder, the anhydrous of which contains 0.1% wt/wt % water. The molecular formula of CX-8998 hydrochloride salt is C20H24F3N202Cl, with a molecular weight of 416.875 g/mol. In some embodiments, CX-8998 is in the form of a base (e.g., CX-8998 free base). In some embodiments, CX-8998 is in the form of a salt (e.g., CX-8998 hydrochloride salt). In some embodiments, CX-8998 is deuterated, in the form of a polymorph, or in the form of a structural isomer of CX-8998 (e.g., CX-8998 tautomer).

An oral dosage form of CX-8998 or a pharmaceutically acceptable salt thereof comprising an immediate release component and a controlled release component is disclosed in U.S. patent application Ser. No. 17/282,730 titled “Treating Essential Tremor Using (R)-2-(4-Isopropylphenyl)-N-(1-(5-(2,2,2-Trifluoroethoxy)Pyridine-2-yl)Ethyl)Acetamide”, the entirety of which is incorporated herein by reference. Description of the oral dosage form and method of making the oral dosage form can be found in, e.g., modified release prototype formulation 2 on page 163 lines 10-11, Examples 26-29, 31, and 33. which are incorporated herein by reference.

### Methods

Methods of treating a movement disorder in an individual in need thereof, comprising administering to the individual an oral dosage form of CX-8998 or a pharmaceutically acceptable salt thereof are provided herein. To achieve treatment, an increasing amount of CX-8998 (e.g., one or more doses of CX-8998) may be given to the individual during a titration period (e.g., 1 week, 2 weeks, 3 weeks, 1 months, 1.5 months, 2 months, etc.). By administrating to the individual an in increasing amount of CX-8998 during a titration period, treatment may be achieved with reduced severity, frequency, and/or duration of adverse events (e.g., dizziness, headache, euphoria, syncope, etc.). An optimal dose (e.g. 10, 15, 20, 25, 30, 35, 40, 45, 50 mg, etc) of CX-8998 (e.g., CX-8998 free base) may be reached by the end of the titration period. The optimal dose may be determined based on one or more factors, such as severity of the movement disorder, severity of adverse events, frequency of adverse events, duration of adverse events, etc. Additionally, treatment may comprise maintaining the administration of CX-8998 (e.g., optimal dose) for a period of time (e.g., 1 to 4 weeks, 2 months, 3 months, 6 months, or 1 year or more) following the last dose administered (e.g., the last dose administered during the titration period).

### Titration Period

In some embodiments, treatment comprises administering to the individual one or more doses (e.g., a first dose, a second dose, a third dose, a fourth dose, a fifth dose, etc.) of an oral dosage form of CX-8998 or a pharmaceutically acceptable salt thereof over a titration period. The titration period may comprise one or more administration periods (e.g., a first administration period, a second administration period, a third administration period, a fourth administration period, a fifth administration period, etc.). The one or more doses may be administered orally to the individual once daily (QD) in the one or more administration periods. For example, a first dose may be administered orally to the individual once daily on each day of the first administration period. A second dose may be administered orally to the individual once daily on each day of the second administration period. A third dose may be administered orally to the individual once daily on each day of the third administration period. A fourth dose may be administered orally to the individual once daily on each day of the fourth administration period. A fifth dose may be administered orally to the individual once daily on each day of the fifth administration period.

In some embodiments, the one or more doses is of an increasing amount of CX-8998 (e.g., the amount of CX-8998 in a subsequent dose is more than the amount of CX-8998 in any one of the previous doses). For example, the first dose may comprise at least 2 mg (e.g., 2, 3, 4, 5, 6, 7, 8 mg, etc.) of CX-8998. The second dose may comprise at least 3 mg (e.g., 3, 4, 5, 6, 7, 8, 9, 10, 11, 12, 13, 14, 15, 16 mg, etc.) of CX-8998. The third dose may comprise at least 4 mg (e.g., 4, 5, 6, 7, 8, 9, 10, 11, 12, 13, 14, 15, 16, 17, 18, 19, 20, 22, 25, 28, 30, 32, 35 mg, etc.) of CX-8998. The fourth dose may comprise at least 5 mg (e.g., 5, 6, 7, 8, 9, 10, 11, 12, 13, 14, 15, 16, 17, 18, 19, 20, 22, 25, 28, 30, 32, 35, 40, 45, 50 mg, etc.) of CX-8998. The fifth dose may comprise at least 6 mg (e.g., 6, 7, 8, 9, 10, 11, 12, 13, 14, 15, 16, 17, 18, 19, 20, 22, 25, 28, 30, 32, 35, 40, 45, 50, 55, 60 mg, etc.) of CX-8998.

In some embodiments, the amount of CX-8998 in a a subsequent dose (e.g. the second, third, fourth, or fifth dose) is at least 2 mg (e.g., 3, 4, 5, 6, 7, 8, 9, 10, 11, 12, 13, 14, 15, 16, 17, 18, 19, 20 mg, etc.) more than the amount of CX-8998 in any one of the previous doses (e.g., the first, second, third, or fourth dose). In some embodiments, the amount of CX-8998 in a subsequent dose (e.g., the second dose) is 5 mg more than the amount of CX-8998 in any one of the previous doses (e.g., the first dose). In some embodiments, the amount of CX-8998 in a subsequent dose (e.g., the third or fourth dose) is 10 mg more than the amount of CX-8998 in any one of the previous doses (e.g., the second or third dose).

In some embodiments, the amount of CX-8998 in a subsequent dose is at least 20% (e.g., 20, 25, 30, 35, 40, 45, 50, 55, 60, 65, 70, 75, 80, 85, 90, 95, 100%) more than the amount of CX-8998 in any one of the previous doses. In some embodiments, the amount of CX-8998 in a subsequent dose is 50% more than the amount of CX-8998 in any one of the previous doses. For example, a subsequent dose may be 15 mg, if any one of the previous doses is 10 mg: a subsequent dose may be 30 mg, if any one of the previous doses is 20 mg.

In some embodiments, the amount of CX-8998 in a subsequent dose is at least 1.5 times (e.g., 1.5, 2, 2.5, 3, 3.5, 4, 4.5, 5, 6 times, etc.) the amount of CX-8998 in any one of the previous doses. In some embodiment, the amount of CX-8998 in a subsequent dose is twice the amount of CX-8998 in any one of the previous doses. For example, a subsequent dose may be 20 mg, if any one of the previous doses is 10 mg. In some embodiment, the amount of CX-8998 in a subsequent dose is 3 times the amount of CX-8998 in any one of the previous doses. For example, a subsequent dose may be 30 mg, if any one of the previous doses is 10 mg. In some embodiment, the amount of CX-8998 in a subsequent dose is 4 times the amount of CX-8998 in any one of the previous doses. For example, a subsequent dose may be 20 mg, if any one of the previous doses is 5 mg. In some embodiment, the amount of CX-8998 in a subsequent dose is 6 times the amount of CX-8998 in any one of the previous doses. For example, a subsequent dose may be 30 mg, if any one of the previous doses is 5 mg.

In some embodiments, each one of the one or more doses comprises one or more (e.g., 1, 2, 3, 4, 5, 6, 7, 8, 9, 10, 11, 12, 13, 14, 15, etc.) unit dose of CX-8998. In some embodiments, the unit dose comprises at least about 2 mg (e.g., 2, 3, 4, 5, 6, 7, 8, 9, 10, 11, 12, 13, 14, 15, 16, 17, 18, 19, 20 mg, etc) of CX-8998. In some embodiment, the first dose contains at least one unit dose of CX-8998. In some embodiment, the second dose contains at least two (e.g., 2, 3, 4, 5, etc.) unit doses of CX-8998. In some embodiment, the third dose contains at least three (e.g., 3, 4, 5, 6, 7, etc.) unit doses of CX-8998. In some embodiment, the fourth dose contains at least four (e.g., 4, 5, 6, 7, 8, etc.) unit doses of CX-8998. In some embodiment, the fifth dose contains at least five (e.g., 5, 6, 7, 8, 9 etc.) unit doses of CX-8998.

In some embodiments, the amount of CX-8998 in each one of the one or more unit dose may be the same or different. For example, the second dose (or the third, fourth, or fifth dose) of about 20 mg of CX-8998 may comprise four 5 mg unit dose (4×5 mg), or one 10 mg unit dose and two 5 mg unit dose (1×10 mg+2×5 mg), or one 5 mg unit dose and one 15 mg unit dose (1×5 mg+1×15 mg). In some embodiments, the second dose (or the third, fourth, or fifth dose) comprises two or more unit doses, wherein the two or more unit doses comprise a same amount of CX-8998. In some embodiments, the second dose (or the third, fourth, or fifth dose) comprises two or more unit doses, wherein the two or more unit doses comprise a different amount of CX-8998.

In some embodiments, the one or more administration periods are initiated consecutively. For example, a second administration period may be initiated following the last dose administered in the first administration period. An optional third administration period may be initiated following the last dose administered in the second administration period. An optional fourth administration period may be initiated following the last dose administered in the third administration period. An optional fifth administration period is initiated following the last dose administered in the fourth administration period.

In some embodiment, each administration period separately comprises at least 3 days (e.g., at least 3, 4, 5, 6, 7, 8, 9, 10, 11, 12, 13, 14 days, or at least 1 week, 2 weeks, or 3 weeks, etc.). In some embodiment, each administration period comprises 7 days or a week. In some embodiments, the titration period comprises at least 2 (e.g., 2, 3, 4, 5, 6, 7, 8, etc.) administration periods. In some embodiments, the titration period comprises at least 7 days or 1 week (e.g., 1.5 weeks, 14 days or 2 weeks, 2.5 weeks, 21 days or 3 weeks, 3.5 weeks, 28 days or 4 weeks, 4.5 weeks, 35 days or 5 weeks, 5.5 weeks, 42 days or 6 weeks, etc.). In some embodiments, the titration period comprises at least 14 days or 2 weeks. In some embodiments, the titration period comprises at least 21 days or 3 weeks. In some embodiments, the titration period may be less than 1 month, about 1 month, or at least 1 month.

The optimal dose may be any one of the doses (e.g., first, second, third, fourth, or fifth dose) administered during the titration period. In some embodiment, the optimal dose is the first dose. In some embodiment, the optimal dose is the second dose. In some embodiment, the optimal dose is the third dose. In some embodiment, the optimal dose is the fourth dose. In some embodiment, the optimal dose is the fifth dose. In some embodiment, the optimal dose is the maximum dose given during the titration period. In some embodiment, the optimal dose is at least 5 mg (e.g., 5, 6, 7, 8, 9, 10 mg, etc.) of CX-8998. In some embodiment, the optimal dose is at least 10 mg (e.g., 10, 11, 12, 13, 14, 15, 16, 17, 18, 19, 20 mg, etc.) of CX-8998. In some embodiment, the optimal dose is at least 20 mg (e.g., 20, 21, 22, 23, 24, 25, 26, 27, 28, 29, 30 mg, etc.). In some embodiment, the optimal dose is at least 30 mg (e.g., 30, 31, 32, 33, 34, 35, 36, 37, 38, 39, 40, 45, 50 etc.). In some embodiment, the optimal dose is 10 mg. In some embodiment, the optimal dose is 20 mg. In some embodiment, the optimal dose is 30 mg.

In some embodiments, treatment comprises maintaining the administration of CX-8998 for a period of time following the last dose. The last dose may be the last dose administered in the first, second, third, fourth, or fifth administration period. The last dose may be the optimal dose. The last dose may be the maximum dose. The period of time following the last dose may be 1 to 4 weeks, 2 months, 3 months, 6 months, or 1 year or more.

### Movement Disorder

In some embodiments, treatment is effective to prevent a movement disorder. For example, treatment may include preventing a movement disorder in an individual who may be predisposed to the movement disorder but does not yet experience or display the pathology or symptomatology of the movement disorder). In some embodiments, treatment is effective to inhibit a movement disorder. For example, treatment may include inhibiting a movement disorder in an individual who is experiencing or displaying the pathology or symptomatology of the movement disorder (i.e., arresting further development of the pathology and/or symptomatology). In some embodiment, treatment is effective to ameliorate a movement disorder. For example, treatment may include ameliorating a movement disorder in an individual who is experiencing or displaying the pathology or symptomatology of the movement disorder (i.e., reversing the pathology and/or symptomatology) such as decreasing the severity of movement disorder or reducing or alleviating one or more symptoms of the movement disorder.

In some embodiments, treatment is effective to reduce or eliminate one or more symptoms of the movement disorder in the individual. Examples of symptoms of movement disorders include, without limitation, tremors (e.g., rhythmic tremors), unsteady gait (e.g., ataxia), dystonic movements, freezing (bradykinesia), tics, spasms, other dyskinetic movements, seizures, pain, sensory problems, psychiatric symptoms, cognitive impairment, hearing impairment, and mood changes. In some embodiments, treatment is effective to reduce or eliminate tremors in the individual. The tremor may include but not limited to essential tremor, Parkinsonian tremor, dystonic tremor, cerebellar tremor, psychogenic tremor, orthostatic tremor, and physiologic tremor. The tremor may be any appropriate type of tremor (e.g., a familial tremor, an action tremor, a postural tremor, a kinetic tremor, and/or a resting tremor). The tremor may affect any appropriate part of a mammal (e.g., hands, head, voice, arms, fingers, legs, chin, and other parts of an individual's body). In some embodiments, treatment is effective to reduce or eliminate tremors in the individual's upper limb.

In some embodiments, treatment is effective to reduce the severity of the movement disorder (e.g., essential tremor) and/or a symptom of a movement disorder in the individual by, for example, 10, 20, 30, 40, 50, 60, 70, 80, 90, 95, or more percent. Any appropriate method may be used to evaluate the severity of a movement disorder and/or a symptom of a movement disorder. In some embodiments, the severity of a movement disorder and/or a symptom of a movement disorder can include evaluating one or more global functional measures. In some embodiments, the severity of a movement disorder and/or a symptom of a movement disorder can include evaluating one or more specific functional measures. Examples of methods that can be used to evaluate the severity of a movement disorder and/or a symptom of a movement disorder include, without limitation, measurements of activities of daily living (e.g., as measured using TETRAS), clinician global impression of improvement (e.g., as measured using CGI-I), patient global impression of change (e.g., as measured using PGIC), tremor specific goal attainment (e.g., as measured using GAS), quality of life (e.g., as measured using QUEST tremor medication satisfaction sub-item), and archimedes spiral (e.g., as measured using pen and paper as in the TETRAS-PS sub-item and/or measured digitally using a tablet and stylus such as in iMotor).

Methods to evaluate the severity of a movement disorder and/or a symptom of a movement disorder are described in, for example, Elble et al, 2013 Movement Disorders, 28 1793: Fahn et al. “Clinical rating Scale for Tremor,” p. 225-34 In: Jankovik J and Tolosa E. Parkinson's Disease and Movement Disorders. 1988 Baltimore-Milnich: Urban & Schwarzenberg; and Haubenberger et al. 2016 Movement Disorders, 31, No. 9; Fahn et al. Recent Developments in Parkinson's Disease, Vol 2. Florham Park, NJ. Macmillan Health Care Information 1987, pp 153-163 and 293-304: Treatment guidelines for essential tremor by the American academy of neurology such as those available at the website aan.com/Guidelines/home/Guideline Detail/492; and Treatment guidelines for Parkinson's disease by the American academy of neurology such as those available at the website movementdisorders.org/MDS-Files 1/Resources/PDFs/TreatmentsforMotorSymptomsofPD-2018.pdf.

### Essential Tremor

In some embodiments, the movement disorder is essential tremor or essential tremor plus. Essential tremor and essential tremor plus may be diagnosed according to the Movement Disorder Society (MDS) Consensus Statement on the Classification of Tremors from the Task Force on Tremor of the International Parkinson's and Movement Disorder Society (Bhatia 2018). For example, criteria for essential tremor may include one or more of the following: 1. isolated tremor syndrome of bilateral upper limb action tremor: 2, at least 3 years' duration: 3. with or without tremor in other locations (eg. head, voice, or lower limbs); and 4, absence of other neurological signs, such as dystonia, ataxia, or parkinsonism. Essential tremor plus may include tremor with the characteristics of ET and additional neurological signs of uncertain significance such as impaired tandem gait, questionable dystonic posturing, memory impairment, or other mild neurologic signs of unknown significance that do not suffice to make an additional syndrome classification or diagnosis. ET with tremor at rest may be classified as essential tremor plus. Exclusion criteria for essential tremor and essential tremor plus may include one or more of the following: isolated focal tremors (e.g., voice or head), orthostatic tremor with a frequency >12 Hz, task- and position-specific tremors, and sudden onset and step-wise deterioration.

In some embodiments, the individual in need of treatment is diagnosed with mild essential tremor. In some embodiments, the individual in need of treatment is diagnosed with moderate to severe essential tremor. In some embodiments, the individual in need of treatment is diagnosed with severe essential tremor. In some embodiment, the individual in need of treatment satisfies one or more of the following: a score of >22 on the TETRAS-ADL, score of >5 on the sum of items 6 and 7 of the TETRAS-PS, and CGI-S rating of at least moderate for ability to function. In some embodiment, the individual in need of treatment satisfies all of the following: a score of ≥22 on the TETRAS-ADL, score of >5 on the sum of items 6 and 7 of the TETRAS-PS, and CGI-S rating of at least moderate for ability to function.

In some embodiments, the individual is a human. In some embodiment, the individual is a juvenile. In some embodiment, the individual is an adult. In some embodiment, the individual is a senior adult. In some embodiments, the individual is a female. In some embodiments, the individual is a male

### Efficacy Assessments

In some embodiments, efficacy of the treatment may be assessed by Tremor Research Group Essential Tremor Rating Assessment Scale (TETRAS). The Tremor Research Group first published TETRAS in 2008 (Elble 2008). TETRAS consists of a 12-item activities of daily living (ADL) subscale, and a 9-item performance subscale (PS). TETRAS was developed as a rapid clinical assessment of ET that requires no equipment other than pen and paper. In some embodiments, efficacy of the treatment may be assessed by Tremor Research Group Essential Tremor Rating Assessment Scale-Activities of Daily Living (TETRAS-ADL) subscale. TETRAS-ADL subscale is a patient-rated scale of the impact of tremor on day-to-day functioning that is administered by a trained interviewer. The TETRAS-ADL directly measures how a patient functions by assessing activities impacted by tremor, such as eating and drinking, dressing and personal hygiene, carrying items, and finer motor skills (Elble 2012). The ADL subscale includes many of the items assessed in previously developed scales for the evaluation of tremor (Fahn 1993: Louis 2000: Bain 1993), including eating and drinking, dressing and personal hygiene, carrying items and finer motor skills. Each item in the TETRAS-ADL is rated on a 0 to 4 scale, with 0 representing normal activity and 4 representing severe abnormality. The sum of the individual scores provides the overall score, ranging from 0 to 48. The TETRAS-ADL has face validity, has preliminarily demonstrated test-retest reliability, and is highly correlated with the TETRAS-PS (Elble 2012: Elble 2016). It has also demonstrated sensitivity to change with CX-8998 treatment in the T-CALM study. In some embodiments, treatment is effective to achieve improvement in TETRAS-ADL subscale.

In some embodiments, efficacy of the treatment may be assessed by Clinical Global Impression of Change (CGI-C). CGI-C is a 5-point Likert-type rating scale and a widely used assessment to assess efficacy in clinical drug trials. CGI-C is clinician-rated, which specifically evaluates the change in patients' ability to function, and complements the patient's perspective, as represented by TETRAS-ADL. Investigators as trained raters rates their impression of any change in the severity of the participant's condition since Baseline on a 5-point scale ranging from 1 (much improved) to 5 (much worse). Investigators rating focuses on the participants change in their ability to function due to ET. In some embodiment, treatment is effective to achieve improvement in CGI-C.

In some embodiments, efficacy of the treatment may be assessed by Tremor Research Group Essential Tremor Rating Assessment Scale-Performance Subscale (TETRAS-PS). TETRAS-PS quantifies tremor in the head, face, voice, limbs, and trunk. TETRAS-PS is a clinical rating scale that has demonstrated sensitivity to change with treatment, and is recommended by the Movement Disorder Society (MDS) Task Force to assess tremor severity (Elble 2013). Each item on the TETRAS-PS is rated on a 0 to 4 rating scale, with scoring of upper limb tremor allowing for 0.5-point increments. Specific amplitude ranges (measured in centimeters) define the tremor rating. Raters first estimate the maximum amplitude of tremor and then assign the corresponding rating. The sum of the individual rating scores provides the overall performance score, ranging from 0 to 64. Administration of the TETRAS-PS takes approximately 10 minutes. The TETRAS-PS has demonstrated both test-retest reliability and sensitivity to change (Elble 2012: Study CX-8998-CLN2-001 [T-CALM]) and was given a “recommended” rating as a tremor severity scale by the MDS (Elble 2013).

In some embodiments, efficacy of the treatment may be assessed by the sum of items 6 and 7 on TETRAS-PS. Items 6 (drawing an Archimedes spiral) and 7 (handwriting) of the TETRAS-PS evaluate the impact of upper limb tremor on performance, and thus represent an objective measure of the impact of upper limb tremor on functional tasks. Both are rated on a 0 (normal) to 4 (severe) rating scale. The Archimedes spiral is tested in both the right and the left hands, while the handwriting is assessed with the dominant hand only. The sum of the TETRAS-PS items 6 and 7 provides a score ranging from 0 to 12. The Archimedes spiral and handwriting tasks have been integral in the routine examination of patients with tremor for decades, heavily utilized in tremor rating scales beyond the TETRAS (Bain 1993; Fahn 1993; Louis 2001), and have demonstrated sensitivity to change with treatment (Calzetti 1982: Haubenberger 2011: Hopfner 2015: Koller 1986; Shill 2004; Tolosa and Loewenson 1975). In some embodiments, treatment is effective achieve improvement in the sum of items 6 and 7 on TETRAS-PS.

In some embodiments, efficacy of the treatment may be assessed by Quality of Life in Essential Tremor Questionnaire (QUEST). The Quality of Life in Essential Tremor Questionnaire (QUEST) was developed to specifically assess the impact of ET on health-related quality of life (Tröster 2005). The QUEST is a 30-item questionnaire comprising 5 subscales (physical, psychosocial, communication, hobbies/leisure, and work/finance) and a total score, plus 3 additional items relating to sexual function and satisfaction with tremor control and medication side effects. Initial reports provide preliminary support of its reliability and validity. The internal consistency was very good to excellent for 4 of the subscales and the total score, and moderately high for the Work/Finance subscale (Tröster 2005). The QUEST has also demonstrated sensitivity to change with deep brain stimulation for ET (Sandvik 2012).

In some embodiments, efficacy of the treatment may be assessed by Patient Global Impression of Change (PGI-C). The PGI-C is a 5-point Likert-type rating scale and a widely used assessment to assess efficacy in clinical drug trials. Participants rates the change in their condition since Baseline on a 5-point scale ranging from 1 (much improved) to 5 (much worse). Participants rating focuses on the change in their ability to function due to ET.

In some embodiments, efficacy of the treatment may be assessed by Clinical Global Impression of Severity (CGI-S). The Clinical Global Impression of Severity (CGI-S) is assessed by qualified medical personnel to assess the severity of participants' ET. The severity assessment rates the participants' ability to function due to their ET. CGI-S is a 5-point Likert-type rating scale and a widely used assessment in clinical psychopharmacology trials to assess severity of illness. The responses to this investigator-completed scale range from 1 (no limitations) to 5 (severe). The investigator rates his/her impression of the severity of the participant's current ability

In some embodiments, efficacy of the treatment may be assessed by a 36-item Short Form Health Survey Version 2. The 36-item Short Form Health Survey Version 2 (SF-36v2) is a multi-purpose, short form health survey with 36 questions. It yields an 8-scale profile of functional health and well-being scores as well as psychometrically-based physical and mental health summary measures and a preference-based health utility index (Hays and Stewart 1992: Ware and Sherbourne 1992).

In some embodiments, efficacy of the treatment may be assessed by Essential Tremor Embarrassment Assessment. The Essential Tremor Embarrassment Assessment (ETEA) is a patient-rated questionnaire administered by a health care provider or researcher that contains 14-items assessing embarrassment related to tremor. Participants provide a simple response (disagree or agree) to each of the 14-items, the sum of which yields an initial score (Score A, range=0 to 14). Participants then provide a more nuanced response to each question on a 0 to 5 point Likert scale ranging from disagree (0) to agree strongly (5). The sum of the nuanced responses yields a second score (Score B, range=0 to 70). Higher scores on both the simple and nuanced responses indicate greater embarrassment. The ETEA was developed based on input from both tremor experts and patients, and was subsequently validated in 75 patients with ET where it demonstrated high internal consistency (Traub 2010). The ETEA has also demonstrated sensitivity to change with treatment in patients with ET (Kreisler 2019).

### Adverse Event

In some embodiments, treatment is effective to reduce the severity, frequency, and/or duration of one or more adverse event. An adverse event (AE) may or may not be treatment-emergent. In some embodiments, an AE is any untoward medical occurrence in a clinical study participant, temporally associated with the use of study intervention, whether or not considered related to the study intervention. An AE can therefore be any unfavorable and unintended sign (including an abnormal laboratory finding), symptom, or disease (new or exacerbated) temporally associated with the use of study intervention. AE may include: 1). Any abnormal laboratory test results (hematology, clinical chemistry, or urinalysis) or other safety assessments (eg. ECG, radiological scans, vital signs measurements), including those that worsen from baseline, considered clinically significant in the medical and scientific judgment of the investigator (ie, not related to progression of underlying disease): 2). Exacerbation of a chronic or intermittent pre-existing condition including either an increase in frequency and/or intensity of the condition: 3). New conditions detected or diagnosed after study intervention administration even though it may have been present before the start of the study; 4) Signs, symptoms, or the clinical sequelae of a suspected drug-drug interaction: 5). Signs, symptoms, or the clinical sequelae of a suspected overdose of either study intervention or a concomitant medication. Overdose per se will not be reported as an AE/SAE unless it is an intentional overdose taken with possible suicidal/self-harming intent. Such overdoses should be reported regardless of sequalae. “Lack of efficacy” or “failure of expected pharmacological action” per se will not be reported as an AE or SAE.

In some embodiments, the one or more adverse event is a treatment-emergent adverse event. All treatment-emergent adverse events are coded into the Medical Dictionary for Regulatory Activities (MedDRA) version 20 with system organ classes and preferred terms and displayed in frequency tables by treatment group. Adverse events are characterized by maximum severity, drug-related adverse events, serious adverse events and adverse events leading to discontinuation of study. The adverse event includes but is not limited to dizziness, headache, euphoria, disturbance in attention, paresthesia, hallucination, insomnia, dry mouth, dysguesia, hypoesthesia, somnolence, lethargy, sleep disturbance, nausea, vomiting, akathisia, decreased level of consciousness, syncope, memory impairment, anxiety, restlessness, fatigue, irritability, constipation, tinnitus, anorexia, emotional disturbances, sexual impotency, diplopia, nystagmus, drowsiness, morbilliform skin eruptions, granulocytopenia, agranulocytosis, red-cell hypoplasia, aplasia, or any combinations thereof.

In some embodiments, the one or more adverse event is a nervous system disorder (e.g., dizziness, headache, disturbance in attention, dysgeusia, paraesthesia, somnolence, hypesthesia, etc.), a psychiatric disorder (e.g., euphoric mood, insomnia, abnormal dreams, hallucination, etc.), a gastrointestinal disorder (e.g., dry mouth, nausea, vomiting, etc.), infections and infestations (e.g., urinary tract infection), or an ear and labyrinth disorder (e.g., tinnitus).

In some embodiments, treatment is effective to avoid occurrence of one or more severe adverse event. Intensity of an adverse event may include mild (e.g., asymptomatic or mild symptoms: clinical or diagnostic observations only), moderate (e.g., minimal, local or noninvasive intervention indicated: limiting age-appropriate instrumental activities of daily living [ADL]), severe (e.g., severe or medically significant but not immediately life-threatening: hospitalization or prolongation of hospitalization indicated: disabling: limiting self-care ADL), life-threatening (e.g., life-threatening consequences: urgent intervention indicated), and fatal (e.g., death related to adverse event).

In some embodiments, the individual experiences no severe adverse event by the end of the first administration period. In some embodiments, the individual experiences no severe adverse event by the end of the second administration period. In some embodiments, the individual experiences no severe adverse event by the end of the third administration period. In some embodiments, the individual experiences no severe adverse event by the end of the fourth administration period. In some embodiments, the individual experiences no severe adverse event by the end of the fifth administration period.

### Administration

In some embodiments, the oral dosage form is administered to the individual once daily (QD). A once daily administration may include administering one or more unit dose simultaneously (e.g., within one minute) or consecutively (e.g., ≥one minute and ≤five minutes). For example, if the fifth dose comprises six unit doses wherein each unit dose is of 5 mg CX-8998, a once daily administration of the fifth dose includes administering all of the six unit doses within five minutes, regardless of whether each unit dose is administered separately.

In some embodiments, the oral dosage form is administered to the individual in a fasted state. For example, the individual may have fasted for at least 4 hours (e.g., 4, 5, 6, 7, 8, 9, 10, 11, 12 hours) prior to administration. In some embodiments, the oral dosage form is administered to the individual without food. In some embodiments, the oral dosage form is administered to the individual with food. In some embodiments, the oral dosage form is administered to the individual in the morning (e.g., any time from about 6 am to about noon). In some embodiments, the oral dosage form is administered to the individual within about 4 hours (e.g., within about 4, 3, 2, or 1 hour) after waking.

Other administration routes may also be used, which include but are not limited to: parenteral (e.g., intravenous, subcutaneous, intramuscular, intraperitoneal, or intrapleural) and transdermal administration routes.

### Examples

### Example 1. A Phase 2b Study to Assess the Safety and Efficacy of CX-8998 in the Treatment of Adults with Moderate to Severe Essential Tremor

This study is a 12-week, double blind, placebo-controlled, randomized, parallel-group, multicenter study of the safety and efficacy of CX-8998 in the treatment of adult participants with essential tremor, as defined by the Movement Disorder Society (MDS) Consensus Statement on the Classification of Tremors from the Task Force on Tremor of the International Parkinson's and MDS.

The total duration of the study for each participant is approximately 21 weeks. During the Screening Period, which occurs over a period of up to 7 weeks, all participants are evaluated for eligibility and will washout any prohibited medications. Participants may be allowed to rescreen once if the rescreening is approved by the medical monitor. Participants eligible for the study are randomized in a 1:1:1:1 ratio to receive once daily doses of 10, 20, or 30 mg CX-8998 or placebo during a 12-week Double-blind Treatment Period. Randomization is stratified by TETRAS-ADL score (≤27 and >27), as assessed at the Baseline Visit. CX-8998 is administered orally (PO) once daily in the morning on an empty stomach for 12 weeks.

Dosing is titrated as follows: participants randomized to the 10 mg/day dose will initially receive 5 mg/day from Day 1 through Day 7, and 10 mg/day starting on Day 8; participants randomized to the 20 mg/day dose will initially receive 5 mg/day from Day 1 through Day 7, 10 mg/day from Day 8 through Day 14, and 20 mg/day starting on Day 15; participants randomized to the 30 mg/day dose will initially receive 5 mg/day from Day 1 through Day 7, 10 mg/day from Day 8 through Day 14, 20 mg/day from Day 15 through Day 21, and 30 mg/day starting on Day 22. Once participants reach their assigned fixed dose, they will continue on that dose for the remainder of the planned 12-week treatment period. No dose adjustments will be allowed. Participants who cannot tolerate their assigned fixed dose of CX-8998 will be withdrawn from the study.

The target patient population is participants with moderate to severe ET. Essential tremor severity level in this study is defined by eligibility criteria on patient-rated (Tremor Research Group Essential Tremor Rating Assessment Scale [TETRAS]-Activities of Daily Living [TETRAS-ADL]), clinician-rated (Clinical Global Impression of Severity [CGI-S]), and objective measures of disability (TETRAS-Performance Subscale [PS] items 6 and 7).

The primary endpoint for the study is the TETRAS-ADL subscale, a patient-rated scale of the impact of tremor on day-to-day functioning administered by a trained interviewer. The TETRAS-ADL directly measures how a patient functions by assessing activities impacted by tremor, such as eating and drinking, dressing and personal hygiene, carrying items, and fine motor skills. The key secondary endpoints are the Clinical Global Impression of Change (CGI-C), and the sum of items 6 and 7 on the TETRAS-PS. The CGI-C specifically evaluates the change in patients ability to function, and adequately complements the patient's perspective, as represented by the TETRAS-ADL.

### Study Population

Inclusion criteria includes the following: 1. participant must be 18 to 80 years of age inclusive, at the time of signing the informed consent: 2. participants who are diagnosed with ET (including ET plus) according to the MDS Consensus Statement on the Classification of Tremors from the Task Force on Tremor of the International Parkinson's and Movement Disorder Society (Bhatia 2018) and centrally reviewed by an Enrollment Adjudication Committee (EAC: see Section 10.1.5): 3. participants have moderate to severe disability associated with tremor at both the Screening and Baseline visits, as determined by all of the following: a, score of >22 on the TETRAS-ADL; b. score of >5 on the sum of items 6 and 7 of the TETRAS-PS (note: The TETRAS-PS is rated by a blinded and trained rater on site); and c. CGI-S rating of at least moderate for participants' ability to function. Participants who are taking any prohibited medications that could impact their tremor assessment (eg, medications for the treatment of tremor, or medications that might produce tremor) at the Screening Visit must return to the clinic for a second Screening Visit (Screening Visit 2). For these participants, the Screening TETRAS-ADL. TETRAS-PS items 6 and 7, and CGI-S assessments that will be used to determine eligibility will be performed at Screening Visit 2 after participants have washed out of their medication.

4. Sex and contraceptive/barrier requirements: participant may be male or female. Male participants are eligible to participate if they agree to the following during the study intervention period and for at least 30 days after the last dose of study intervention: a. refrain from donating sperm, and b, satisfy either one of the following two requirements: i) be abstinent from heterosexual intercourse as their preferred and usual lifestyle (abstinent on a long-term and persistent basis) and agree to remain abstinent; or ii) must agree to use contraception/barrier as detailed below: agree to use a male condom with female partner use of an additional highly effective contraceptive method with a failure rate of <1% per year when having sexual intercourse with a woman of childbearing potential (WOCBP) who is not currently pregnant; agree to use a male condom when engaging in any activity that allows for passage of ejaculate to another person. A female participant is eligible to participate if she is not pregnant or breastfeeding, and one of the following conditions applies: is a woman of non-childbearing potential (WONCBP); or is a WOCBP and using a contraceptive method that is highly effective, with a failure rate of <1% during the study intervention period and for at least 30 days after the last dose of study intervention. The investigator should evaluate the potential for contraceptive method failure (eg, noncompliance, recently initiated) in relationship to the first dose of study intervention. A WOCBP must have a negative highly sensitive pregnancy test (urine or serum as required by local regulations) at Screening and at the Baseline Visit before the first dose of study intervention. If a urine test cannot be confirmed as negative (eg, an ambiguous result), a serum pregnancy test is required. In such cases, the participant must be excluded from participation if the serum pregnancy result is positive. The investigator is responsible for review of medical history, menstrual history, and recent sexual activity to decrease the risk for inclusion of a woman with an early undetected pregnancy. 5. Participants must be capable of giving signed informed consent; and 6. Participants must be willing and able to comply with the study design schedule and other requirements.

Exclusion criteria includes the following: Medical conditions: 1. female participants who are pregnant, nursing, or lactating, or plan to become pregnant during the study or within 90 days of study completion: 2. known history or current evidence of other medical or neurological conditions that may cause or explain the participant's tremor in the opinion of the investigator or central reviewer (when applicable), including, but not limited to: Parkinson's disease or features of atypical parkinsonism; psychogenic tremor; clinically significant symptoms or signs of dystonia, myoclonus, or ataxia; cerebellar disease other than ET; traumatic brain injury; alcohol abuse or withdrawal; mercury poisoning; hyperthyroidism; pheochromocytoma; head trauma or cerebrovascular disease within 3 months before onset of ET; multiple sclerosis; clinically significant polyneuropathy in the opinion of the investigator, or; family history or diagnosis of Fragile X syndrome: 3, considered at risk of falls in the opinion of the investigator; 4. has evidence at Screening of severe cognitive impairment as defined by a Montreal Cognitive Assessment (MoCA; score<23), or has cognitive impairment that in the opinion of the investigator would prevent completion of study procedures or the ability to provide informed consent; 5. history or presence of any acutely unstable medical condition, malignancy other than basal cell carcinoma or resected noninvasive cutaneous squamous cell carcinoma, or surgical history that could affect the safety of the participant or interfere with study efficacy, safety, or PK assessments; or the ability of the participant to complete the trial per the judgment of the investigator; 6. history or presence of gastrointestinal (including prior bariatric bypass surgery), hepatic (including alanine aminotransferase [ALT] or aspartate aminotransferase [AST]≥2× upper limit of normal [ULN]), or renal disease (total bilirubin≥1.5 ULN), or any other condition that, in the opinion of the investigator, may interfere with absorption, distribution, metabolism, or excretion of drugs: 7. presence of significant cardiovascular disease at Screening including but not limited to the following: myocardial infarction within the past year: unstable angina pectoris; symptomatic congestive heart failure (American College of Cardiology/American Heart Association stage C or D); revascularization procedures within the past year: ventricular cardiac arrhythmias requiring automatic implantable cardioverter defibrillator or medication therapy: uncontrolled hypertension, or systolic blood pressure ≥155 mmHg or diastolic blood pressure ≥95 mmHg (based on the average of triplicate assessments at Baseline); clinically significant ECG abnormality per the investigator assessment, or Fridericia's corrected QT interval (QTcF)>450 msec for men and >470 msec for women, based on the average of triplicate assessments at Screening or Baseline; or any history of cardiovascular disease or any significant cardiovascular condition that in the investigator's opinion may jeopardize participant safety in the study: 8. history or presence of bipolar and related disorders, schizophrenia, schizophrenia spectrum disorders, or other psychotic disorders according to the Diagnostic and Statistical Manual of Mental Disorders. 5th edition (DSM-5) criteria: 9, current suicidal risk as determined from history, by presence of active suicidal ideation as indicated by positive response to item 4 or 5 on the C-SSRS (within the past 24 months), or any history of suicide attempt: current or past (within 1 year) major depressive episode according to DSM-5 criteria. Participants with stable treated depression are allowed per the judgment of the investigator or the treating medical practitioner and the antidepressant treatment has to be stable for at least 6 months prior to Screening and remain stable for the duration of the study: 10. history (within past 2 years at screening) or presence of substance use disorder (including alcohol) according to DSM-5 criteria, known drug dependence, or seeking treatment for alcohol or substance abuse related disorder. Nicotine use disorder is excluded if it impacts tremor.

Exclusion criteria also includes prior/concomitant therapy: 11. prior magnetic resonance (MR)-guided focused ultrasound, surgical intervention (eg, deep brain stimulation, ablative thalamotomy, gamma knife thalamotomy), or inability to refrain from using a device for treatment of tremor for the duration of the treatment period: 12, botulinum toxin injection in the 6 months before screening or planned use at any time during the study: 13, treatment with any medication that could affect the evaluation of tremor within 2 weeks or 5 half-lives (whichever is longer) before the evaluation of tremor at Screening (Screening Visit 1 or 2, as applicable) or planned use at any time during the study, including: a, medication for the treatment of tremor, unless these medications are being utilized for non-tremor indications (eg. propranolol prescribed for hypertension is allowed). Use of cannabinoids (including CBD) is not permitted recreationally or medically at Screening or throughout the duration of the study (note: if on primidone at Screening, treatment must be discontinued at least 4 weeks prior to Screening Visit 2): b, medication that might produce tremor or interfere with the evaluation of tremor. Note: Regular use of a benzodiazepine, sleep medication or anxiolytic to improve sleep or anxiety is permitted, provided it will continue to be used regularly and at stable doses throughout the study: 14. use of prescription or nonprescription drugs or other products known to be inducers of CYP3A4 or CYP2C9, which cannot be discontinued at least 4 weeks before baseline, or planned use at any time during the study: 15. use of prescription or nonprescription drugs, or other products (eg. grapefruit, grapefruit juice, or Seville oranges) known to be strong or moderate inhibitors of CYP3A4 or CYP2C9, that cannot be discontinued 2 weeks or 5 half-lives, whichever is longer, before baseline or planned use at any time during the study: 16. use of proton pump inhibitors and histamine-2 receptor antagonists, which cannot be discontinued at least 2 weeks before Baseline. or planned use at any time during the study (occasional use of antacids will be permitted, but antacids should be taken at least 4 hours apart from study intervention): 17. inability to refrain from use of medication/substance(s) that might produce tremor or interfere with the evaluation of tremor on study visit days, such as, but not limited to, stimulant decongestants, beta-agonist bronchodilators, and alcohol. Participants who consume caffeine or use tobacco should take their regular amount of caffeine or tobacco on the clinic days.

Exclusion criteria also includes: 18. received an investigational drug 30 days or 5 half-lives prior to the Baseline visit (whichever is longer), or plans to use an investigational drug (other than the study intervention) during the study: 19. received any study intervention in a previous CX-8998 (formerly known as MK-8998) clinical study: 20, a fall in blood pressure (ie, a decrease in systolic blood pressure ≥20 mm Hg or diastolic blood pressure ≥10 mm Hg), or a heart rate increase (ie, >30 beats per minute) observed on the average of the triplicate orthostatic assessments at Baseline, or in the opinion of the investigator, the participant was symptomatic for orthostatic hypotension: 21. laboratory value(s) at screening outside the laboratory reference range that is (are) considered clinically significant by the investigator (clinical chemistry, hematology, and urinalysis) (note: screening laboratory tests may be repeated once): 22. urine drug screen positive at Screening for drugs of abuse (eg, phencyclidine [PCP], cocaine, cannabinoids, opiates, barbiturates, amphetamines, methadone, or MDMA [Ecstasy]) unless explained by use of an allowed prescription medication (eg, benzodiazepine as outlined in exclusion criterion 13b). If the interpretation of positive results is ambiguous, or there are extenuating circumstances, a repeat urine drug screen may be performed if approved by the investigator and the Medical Monitor; 23. regular use of more than 3 units of alcohol per day (see also Exclusion Criterion 17). A unit of alcohol is defined as a 12-fluid ounce (350 mL) glass of beer (5% alcohol by volume), a 5-fluid ounce (150 mL) glass of wine (12% alcohol by volume), or a 1.5 fluid ounce (44 mL) glass of spirit (40% alcohol by volume): 24. regular consumption of caffeine >400 mg/day or >4 cups of coffee per day: 25, allergy or sensitivity to any ingredients in the study intervention formulation or placebo: 26, any other condition and/or situation that causes the investigator or medical monitor to deem a participant unsuitable for the study.

### Example 2. Formulation of Modified Released Form of CX-8998

In one experiment, formulations containing CX-8998 hydrochloride salt are as shown in Table 1. Weight percent (% w/w) is based on a 10 mg unit dose. Modified release (MR) formulation of CX-8998 has an immediate release (IR) component and a delayed release component (DR). Delayed release of CX-8998 is achieved by application of a pH-sensitive coating that is targeted to dissolve at pH=6 (MR1 or MR1′) or at pH=7 (MR2) on an immediate release core.

TABLE 1

| | | MR1 | | MR1′ | | MR2 | |
|---|---|---|---|---|---|---|---|
| | | (60% IR + 40% | | (40% IR + 60% | | (40% IR + 60% | |
| | | MR-pH6) | | MR-pH6) | | MR-pH7) | |
| | | mg/unit | | mg/unit | | mg/unit | |
| | Excipient | dose | % w/w | dose | % w/w | dose | % w/w |
| Core | CX-8998 HCL | 10.96 | 3.84 | 10.96 | 3.70 | 10.96 | 3.70 |
| Bead | Lactose | 126.36 | 44.26 | 126.36 | 42.68 | 126.36 | 42.68 |
| | Monohydrate 312 | | | | | | |
| | Crospovidone | 54.79 | 19.19 | 54.79 | 18.51 | 54.79 | 18.51 |
| | XL-10 | | | | | | |
| | Citric Acid | 17.53 | 6.14 | 17.53 | 5.92 | 17.53 | 5.92 |
| | Anhydrous | | | | | | |
| | Sodium Lauryl | 4.38 | 1.54 | 4.38 | 1.48 | 4.38 | 1.48 |
| | Sulfate | | | | | | |
| | Hydroxypropyl | 4.38 | 1.54 | 4.38 | 1.48 | 4.38 | 1.48 |
| | Cellulose | | | | | | |
| | Butylated | 0.55 | 0.19 | 0.55 | 0.19 | 0.55 | 0.19 |
| | Hydroxyanisole | | | | | | |
| | Butylated | 0.22 | 0.08 | 0.22 | 0.07 | 0.22 | 0.07 |
| | Hydroxytoluene | | | | | | |
| Clear | Opadry Clear | 43.84 | 15.35 | 43.84 | 14.81 | 43.84 | 14.81 |
| Coat | 20A19301 | | | | | | |
| pH 6 | Eudragit L100 | 6.58 | 2.30 | 9.86 | 3.33 | — | — |
| Coat | Eudragit S100 | 6.58 | 2.30 | 9.86 | 3.33 | — | — |
| | Triethyl Citrate | 1.32 | 0.46 | 1.97 | 0.67 | — | — |
| | Talc | 8.00 | 2.80 | 11.34 | 3.83 | 1.48 | 0.50 |
| pH 7 | Eudragit FS 30 D | — | — | — | — | 28.69 | 9.69 |
| Coat | Plasacryl T20 | — | — | — | — | 2.87 | 0.97 |

| | | | | | | |
|---|---|---|---|---|---|---|
| Total | 285.48 | 100.00 | 296.06 | 100.00 | 296.06 | 100.00 |

### Other Embodiments

This application refers to various issued patent, published patent applications, journal articles, and other publications, each of which is incorporated herein by reference.

The foregoing has been described of certain non-limiting embodiments of the present disclosure. Those of ordinary skill in the art will appreciate that various changes and modifications to this description may be made without departing from the spirit or scope of the present disclosure, as defined in the following claims.

What is claimed is:

1. A method of treating a movement disorder in an individual in need thereof, comprising:
a) administering to the individual a first dose of an oral dosage form of CX-8998 or a pharmaceutically acceptable salt thereof, wherein the first dose comprises about 5 mg of CX-8998 and wherein the first dose is administered orally to the individual once daily (QD) on each day of week 1;
b) administering to the individual a second dose of an oral dosage form of CX-8998 or a pharmaceutically acceptable salt thereof, wherein the second dose comprises about 10 mg of CX-8998 and wherein the second dose is administered orally to the individual once daily (QD) on each day of week 2; and
c) optionally administering to the individual a third dose of an oral dosage form of CX-8998 or a pharmaceutically acceptable salt thereof, wherein the third dose comprises about 20 mg of CX-8998 and wherein the third dose is administered orally to the individual once daily (QD) on each day of week 3.

2. The method of claim 1, wherein the method comprises
a) administering the first dose to the individual once daily (QD) on each day of week 1;
b) administering the second dose to the individual once daily (QD) on each day of week 2; and
c) maintaining the administration of the 10 mg of CX-8998 to the individual once daily (QD) for a period of time following the last dose administered in week 2.

3. The method of claim 1, wherein the method comprises a) administering the first dose to the individual once daily (QD) on each day of week 1;
b) administering the second dose to the individual once daily (QD) on each day of week 2;
c) administering the third dose to the individual once daily (QD) on each day of week 3; and
d) maintaining the administration of the 20 mg of CX-8998 to the individual once daily (QD) for a period of time following the last dose administered in week 3.

4. The method of claim 1, wherein the method comprises
a) administering the first dose to the individual once daily (QD) on each day of week 1;
b) administering the second dose to the individual once daily (QD) on each day of week 2;
c) administering the third dose to the individual once daily (QD) on each day of week 3; and
d) administering to the individual a fourth dose of an oral dosage form of CX-8998 or a pharmaceutically acceptable salt thereof, wherein the fourth dose comprises about 30 mg of CX-8998 and wherein the fourth dose is administered orally to the individual once daily (QD) on each day of week 4.

5. The method of claim 4, wherein the method comprises maintaining the administration of the 30 mg of CX-8998 to the individual once daily (QD) for a period of time following the last dose administered in week 4.

6. The method of any one of claim 2, 3 or 5, wherein the period of time is a period of at least 1-4 weeks, 6 months, or 1 year or more.

7. The method of claim 3, wherein the individual experiences no severe adverse event (AE) by the last day of week 2 after the first dose is administered, wherein the adverse event is selected from the group consisting of headache, lightheadedness, dizziness, somnolence, lethargy, impaired concentration, vivid dreams, euphoric mood, elevated mood, racing thoughts, visual hallucination, and syncope.

8. The method of claim 3 or 7, wherein the individual experiences mild adverse event (AE), moderate AE, or no AE by the last day of week 2 after the first dose is administered.

9. The method of claim 4, wherein the individual experiences no severe adverse event (AE) by the last day of week 3 after the first dose is administered, wherein the adverse event is selected from the group consisting of headache, lightheadedness, dizziness, somnolence, lethargy, impaired concentration, vivid dreams, euphoric mood, elevated mood, racing thoughts, visual hallucination, and syncope.

10. The method of claim 4 or 9, wherein the individual experiences mild adverse event (AE), moderate AE, or no AE by the last day of week 3 after the first dose is administered.

11. The method of any one of claims 1-10, wherein the movement disorder is essential tremor, epilepsy, or Parkinson's disease.

12. The method of any one of claims 1-11, wherein the individual is diagnosed with moderate to severe essential tremor.

13. The method of any one of claims 1-12, wherein the individual is diagnosed with severe essential tremor.

14. The method of any one of claims 1-13, wherein the individual is an adult.

15. The method of any one of claims 1-14, wherein the oral dosage form is administered to the individual in a fasted state.

16. The method of any one of claims 1-15, wherein the oral dosage form is administered to the individual in the morning or within about 4 hours after waking.
